(19) United States
(12) Patent Application Publication
(10) Pub. No.: US 2021/0137778 A1
(43) Pub. Date: May 13, 2021

(54) System and Method for Generating, and Delivering to Standing Users, Therapeutic Acoustic Vibrations

(71) Applicant: Vibragenix, LLC, Richland, WA (US)

(72) Inventors: Carlton M. Cadwell, Richland, WA (US); Caroline M. Stites, Richland, WA (US)

(21) Appl. No.: 17/094,496

(22) Filed: Nov. 10, 2020

Related U.S. Application Data
(60) Provisional application No. 62/934,827, filed on Nov. 13, 2019.

(51) Int. Cl. A61H 23/02; A61M 16/06

(52) U.S. Cl. CPC A61H 23/0236; H04R 9/06; A61M 16/0672

(57) ABSTRACT

A system of delivering multi-frequency acoustic vibrations into the body of a user for increasing a voltage of the cells of the user's body is provided. The system including: a base assembly including a space defined by a top plate and a bottom plate including voice coils, magnets and springs, wherein each voice coil is aligned with a magnet and the springs maintain a predefined thrust between the top and bottom plate, each voice coil lying within a magnetic flux of a magnet for producing acoustic waves of predefined frequencies for deliverance into the user's body through the user's feet when the user is standing on the base assembly; and a tower assembly tethered with the base assembly, and including at least one speaker, and one or more cell exciter pads for attaching with a portion of the user's body, the speaker and pads producing acoustic waves of predefined frequencies for deliverance into the user's body.

### CROSS-REFERENCE

The present specification relies on, for priority, U.S. Patent Provisional Application No. 62/934,827, titled “System and Method for Generating, and Delivering to Standing Users, Therapeutic Acoustic Vibrations”, filed on Nov. 13, 2019, which is herein incorporated by reference in its entirety.

### FIELD

The present specification generally relates to therapeutic acoustic systems. More specifically the present specification relates to systems and methods for generating and delivering therapeutic acoustic frequencies to a user for the purposes of enhancing health and wellness and/or re-energizing cells within a user's body.

### BACKGROUND

In a human body, the electrical potential of the cell is one indicator of the overall health of the body. When the electrical potential is low, the cells lose their vitality and ability to function properly, which can lead to fatigue, lack of vitality, and decreased immunity. A live cell blood sample of an unhealthy person under a microscope shows the blood cells clumped together which can be indicative of low electrical potential in the cells. On the other hand, healthy cells having a greater cell potential display a greater degree of motility and a greater degree of separation from other cells.

The energy level or health of a cell within the human body can be measured in millivolts. Healthy cells may have cell voltages of 70 to 90 millivolts with an overall negative charge. Due to the constant stresses of modern life, a toxic diet and/or a toxic environment, cell voltage tends to drop, becoming even more depleted with age and sickness. Another major cause of overall vitality loss is stagnation of lymphatic and other body fluids. Hence, there is a requirement of stimulating circulation of blood within the human body to facilitate detoxification, increase cell voltages, and enhance the flow of energy within the body.

Various mechanisms for delivering vibrations to a human body are available. However, a simple mechanical vibration of the cells does not cause an increase of cellular voltage. Typically, acoustic waves having different frequencies are used to excite certain cells within the body so that the voltage in those specific cells is increased to a healthier level. Conventional acoustic therapy devices that deliver acoustic waves to a standing user are often limited in the extent and range of frequencies they can deliver. Specifically, conventional systems fail to generate frequencies below 30 Hz and above 70 Hz, fail to generate multiple different ranges of frequencies thereby limiting the type of beneficial outcomes available, are unable to accommodate a wide range of user weights, and are unable to combine multiple different frequencies to enable more complex stimulation of specific cells.

Hence, there is need for a system and method of delivering multiple acoustic frequencies simultaneously to a human body. There is also a need for increasing the cell voltages within the body, stimulating circulation of blood, and the lymphatic system to facilitate detoxification of the body. There is a need for an acoustic frequency device that can deliver, to a standing user, frequencies below 30 Hz and greater than 70 Hz and can concurrently generate, and combine, multiple different frequencies. Furthermore, there is a need for an acoustic frequency delivery device that can accommodate a wide range of weights of standing users. There is also a need for an acoustic frequency delivery device that is configured to enable a user to easily select a specific frequency panel and, in response, generate polychromatic, multi-tonal acoustic frequencies.

### SUMMARY

The following embodiments and aspects thereof are described and illustrated in conjunction with systems, tools and methods, which are meant to be exemplary and illustrative, not limiting in scope.

In some embodiments, the present specification discloses a system configured to deliver multi-frequency acoustic vibrations to cells of a user's body, the system comprising: a base assembly comprising a top plate, a bottom plate, a space defined by the top plate and the bottom plate, one or more voice coils positioned in the space, one or more magnets positioned in the space, and one or more springs positioned in the space, wherein the one or more springs is configured to maintain a predefined thrust range between the top plate and the bottom plate, wherein each of the one or more voice coils is aligned with one of the one or more magnets such that the one or more voice coils lies within a magnetic flux of the one or more magnets to thereby produce a first portion of the multi-frequency acoustic vibrations; a vertically extending assembly tethered to the base assembly, wherein the vertically extending assembly comprises at least one speaker configured to produce a second portion of the multi-frequency acoustic vibrations; and one or more cell exciter pads coupled with the vertically extending assembly and configured to attach to a portion of the user's body to deliver a third portion of the multi-frequency acoustic vibrations.

Optionally, the acoustic vibrations comprise acoustic waves of frequencies ranging from 4 to 20,000 Hz.

Optionally, the acoustic vibrations comprise polychromatic and multi-tonal acoustic frequencies.

Optionally, the system further comprises a casing enclosing the top plate the bottom plate, wherein the casing is adapted to receive a user standing thereon.

Optionally, the top plate is coupled with the bottom plate through a plurality of shafts and bearings.

Optionally, the one or more magnets comprise a first magnet, a second magnet, and a third magnet coupled to the top plate, wherein the third magnet is larger than the first magnet and the second magnet, wherein the one or more voice coils comprise a first voice coil, a second voice coil, and a third voice coil coupled to the bottom plate, and wherein the third voice coil is larger than the first voice coil and the second voice coil.

Optionally, the one or more magnets comprise only two magnets, wherein the two magnets are coupled with the top plate, wherein the one or more voice coils comprise only two voice coils, and wherein the two voice coils are coupled with the bottom plate.

Optionally, the one or more springs comprise two or more springs each having a different diameter and a different length to thereby provide a variable thrust based on the user's weight. Optionally, the top plate and the bottom plate comprise steel and have a thickness of 3/16 inches.

Optionally, the top plate and bottom plate are each made of at least one of stainless steel, aluminum, bronze, brass, titanium, or tin.

Optionally, each of the top plate and the bottom plate comprise a rim extending all along edges of the top plate and the bottom plate and has a thickness ranging from 3/16 to ⅛ inches.

Optionally, the vertically extending assembly further comprises a casing enclosing said vertically extending assembly, wherein an outer surface of the casing comprises a reflective material adapted to reflect the second portion of the multi-frequency acoustic vibrations produced by the vertically extending assembly back to the user.

Optionally, the vertically extending assembly is tethered to the base assembly by one or more dampening feet extending on a first side below the base assembly and on a second side being coupled with the vertically extending assembly.

Optionally, the system further comprises a computing device comprising an audio chip coupled with an amplifier coupled with the at least one speaker and the one or more cell exciter pads adapted to enable the user to select a treatment mode from among a plurality of treatment modes, and wherein the computing device is configured to cause the audio chip to generate the multi-frequency acoustic vibrations based upon said selection.

Optionally, the vertically extending assembly further comprises a user interface enabling the user to make the selection.

Optionally, the plurality of treatment modes comprises at least one of a weight loss mode, a pain management mode, an anti-aging mode, a health and fitness mode, or an enhancing sports performance mode.

Optionally, the plurality of treatment modes comprises at least seven hundred predefined combinations of the multi-frequency acoustic vibrations.

Optionally, the system further comprises a sound pod enclosing the base assembly and the vertically extending assembly, wherein the sound pod comprises a roof, enclosing walls and a door adapted to allow the user to enter the sound pod, wherein at least the walls and door comprise a transparent, non-resonating material.

Optionally, the roof comprises a non-resonating material.

Optionally, the walls and door comprise double-walled polycarbonate panels supported by aluminum frames.

Optionally, the system further comprises an oxygen source, wherein the sound pod comprises an inlet adapted to receive oxygen from the oxygen source, and a nasal cannula adapted to direct the oxygen to the user.

Optionally, the sound pod further comprises a fan and a plurality of air vents, wherein the plurality of air vents are positioned in a base of at least one of the walls and wherein the fan is positioned on or in the roof.

In some embodiments, the present specification discloses a method of delivering multi-frequency acoustic vibrations into a body of a user, the method comprising: providing an acoustic generation system, comprising: a base assembly comprising a top plate, a bottom plate, a space defined by the top plate and the bottom plate, one or more voice coils positioned in the space, one or more magnets positioned in the space, and one or more springs positioned in the space, wherein the one or more springs is configured to maintain a predefined thrust range between the top plate and the bottom plate, wherein each of the one or more voice coils is aligned with one of the one or more magnets such that the one or more voice coils lies within a magnetic flux of the one or more magnets to thereby produce a first portion of the multi-frequency acoustic vibrations; a vertically extending assembly tethered to the base assembly, wherein the vertically extending assembly comprises at least one speaker configured to produce a second portion of the multi-frequency acoustic vibrations; and one or more cell exciter pads coupled with the vertically extending assembly and configured to attach to a portion of the user's body to deliver a third portion of the multi-frequency acoustic vibrations; instructing the user to stand on the base assembly; selecting a treatment mode from among a plurality of treatment modes using an interface in data communication with the acoustic generation system; generating one or more of the multi-frequency acoustic vibrations based on the selected treatment mode; and applying the one or more of the multi-frequency acoustic vibrations to the user's feet via the base assembly, the user's body through air from the at least one speaker, or the user's body via the one or more cell exciter pads attached to a portion of the user's body.

Optionally, the multi-frequency acoustic vibrations comprise acoustic of frequencies ranging from 4 to 20,000 Hz.

In some embodiments, the present specification discloses a system for delivering multi-frequency acoustic vibrations to a user for increasing a voltage of the cells of the user's body, the system comprising: a base assembly comprising a space defined by a top plate and a bottom plate, the space comprising one or more voice coils, one or more magnets, and one or more springs, wherein each voice coil is aligned with a magnet and the one or more springs maintain a predefined thrust range between the top and bottom plate, each voice coil lying within a magnetic flux of a magnet for producing acoustic waves of predefined frequencies for deliverance into the user's body through the user's feet when the user is standing on the base assembly; and a tower assembly tethered with the base assembly, the tower assembly comprising at least one speaker for producing acoustic waves of predefined frequencies for deliverance into the user's body; and one or more cell exciter pads coupled with the tower assembly for attaching with a portion of the user's body for delivering acoustic waves of predefined frequencies into the body.

Optionally, the acoustic vibrations comprise acoustic waves of frequencies ranging from 4 to 20,000 Hz.

Optionally, the acoustic vibrations comprise polychromatic and multi-tonal acoustic frequencies.

Optionally the system further comprises a casing enclosing the top plate the bottom plate, wherein the user stands on the casing for receiving the acoustic vibrations.

Optionally, the top plate is coupled with the bottom plate by using shafts and bearings.

Optionally, the one or more magnets comprise two smaller magnets and one larger magnet coupled with the top plate, and the one or more voice coils comprise two smaller voice coils and one larger voice coil coupled with the bottom plate.

Optionally, the one or more magnets comprise two smaller magnets coupled with the top plate, and the one or more voice coils comprise two smaller voice coils coupled with the bottom plate.

Optionally, the one or more springs comprising springs of differing diameter and differing lengths for providing variable thrust based on the user's weight.

Optionally, the top and bottom plates are made of steel and have a thickness of 3/16 inches.

Optionally, the top and bottom plates are made of one of: stainless steel, aluminum, bronze, brass, titanium, and tin.

Optionally, the each of the top and bottom plates have a rim extending all along the edges of the plates and having a thickness ranging from 3/16 to ⅛ inches.

Optionally, the tower assembly further comprises a casing enclosing said assembly, an outer surface of the casing being made of a reflective material for enhancing acoustic delivery to the user by reflecting the acoustic frequencies produced by the tower assembly back to the user.

Optionally, the tower assembly is tethered to the base assembly by using one or more dampening feet extending on a first side below the base assembly and on a second side being coupled with the tower assembly.

Optionally, the system further comprises a computing device comprising an audio chip coupled with an amplifier coupled with the at least one speaker and the one or more cell exciter pads for enabling the user to select a treatment mode from among a plurality of treatment modes, said selection causing the audio chip to generate a predefined range of frequencies corresponding to the selection to be delivered to the user's body.

Optionally, the tower assembly further comprising a user interface enabling the user to make the selection.

Optionally, the plurality of treatment modes comprises one or more of: a weight loss mode, a pain management mode, an anti-aging mode, a health and fitness mode, and an enhancing sports performance mode.

Optionally, the plurality of treatment modes comprises at least seven hundred predefined combinations of deliverable acoustic frequencies.

Optionally, the system further comprises a sound pod enclosing the base assembly and the tower assembly, the sound pod comprising enclosing walls having a door for entering the pod and a roof, wherein the walls, and door are made of a transparent, non-resonating material.

Optionally, the roof is made of a non-resonating material.

Optionally, the walls and door of the system are made of double-walled polycarbonate panels supported by aluminum frames.

Optionally, the sound pod comprises an inlet for importing oxygen into the pod, from an external oxygen source, the imported oxygen being inhaled by the user via a nasal cannula provided within the pod.

Optionally, the sound pod further comprises air vents placed into the base of the enclosing walls and a fan positioned on the roof.

In some embodiments, the present specification discloses a method of delivering multi-frequency acoustic vibrations into the body of a user for increasing a voltage of the cells of the user's body by using a system comprising: a base assembly comprising a space defined by a top plate and a bottom plate, the space comprising one or more voice coils, one or more magnets, and one or more springs, wherein each voice coil is aligned with a magnet and the springs maintain a predefined thrust between the top and bottom plate, each voice coil lying within a magnetic flux of a magnet for producing acoustic waves of predefined frequencies for deliverance into the user's body; and a tower assembly tethered with the base assembly, the tower assembly comprising at least one speaker for producing acoustic waves of predefined frequencies for deliverance into the user's body and one or more cell exciter pads for attaching with a portion of the user's body for delivering acoustic waves of predefined frequencies into the body; the method comprising: standing on the base assembly; selecting a treatment mode from among a plurality of treatment modes; receiving acoustic vibrations of predefined frequencies corresponding to the selection from the base assembly, the acoustic vibrations entering the user's body via feet of the user; receiving acoustic vibrations of predefined frequencies corresponding to the selection from the at least one speaker, the acoustic vibrations travelling through air and entering the user's body; attaching the one or more cell exciter pads with a portion of the user's body; and receiving acoustic vibrations of predefined frequencies corresponding to the selection from the one or more cell exciter pads.

Optionally, the acoustic vibrations comprise acoustic of frequencies ranging from 4 to 20,000 Hz.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates a system for delivering acoustic vibrations to a standing human patient, in accordance with an embodiment of the present specification;

FIG. 2A is a perspective break-away view of the base assembly shown in FIG. 1, in accordance with an embodiment of the present specification;

FIG. 2B illustrates another view of the base assembly shown in FIG. 2A, in accordance with an embodiment of the present specification;

FIG. 2C is a block diagram illustrating the field forces optimizing the coupling of a voice coil and a magnet in the base assembly shown in FIG. 2A, in accordance with an embodiment of the present specification;

FIG. 3A illustrates a base assembly of the system for delivering acoustic vibrations shown in FIG. 1, in accordance with another embodiment of the present specification;

FIG. 3B illustrates another view of the base assembly shown in FIG. 3A, in accordance with an embodiment of the present specification;

FIG. 4A is a perspective view of the tower assembly of the system for delivering acoustic vibrations shown in FIG. 1, in accordance with an embodiment of the present specification;

FIG. 4B is a back view of the tower assembly shown in FIG. 4A, in accordance with an embodiment of the present specification;

FIG. 5 illustrates a sound pod in accordance with an embodiment of the present specification;

FIG. 6 is a flowchart illustrating a method for delivering multi-frequency acoustic vibrations to a user for increasing a voltage of the cells within a user's body, in accordance with an embodiment of the present specification;

FIG. 7A is a screenshot of a selection screen presented to a user for selecting a frequency mode from among a plurality of options provided, in accordance with an embodiment of the present specification;

FIG. 7B illustrates a screen that is presented to the user upon selecting an option ‘manual’ for selecting a frequency mode from among a plurality of options provided, in accordance with an embodiment of the present specification;

FIG. 7C illustrates a screen that is presented to the user upon selecting an option ‘essentials’ for selecting a frequency mode from among a plurality of options provided, in accordance with an embodiment of the present specification;

FIG. 7D illustrates a screen 790 that is presented to the user upon selecting an option ‘sports/recovery’ for selecting a frequency mode from among a plurality of options provided, in accordance with an embodiment of the present specification; and

FIG. 7E illustrates a screen 7020 that is presented to the user upon selecting an option ‘sports/recovery’ for selecting a frequency mode from among a plurality of options provided, in accordance with an embodiment of the present specification.

### DETAILED DESCRIPTION

The present specification provides a system and method for delivering acoustic waves into a human body, thus causing vibrational activity within the body and increasing the cell voltage of each cell of the body. The acoustic, or sound, vibrations delivered to a human body in accordance with embodiments of the present specification enable at least one cell within the body to be re-energized, and thus, regain enough cell voltage to operate more effectively and enhance overall health of the body, starting at a cellular level. Further, the acoustic, or sound, vibrations delivered to a human body causes blood cells to separate from the other blood cells, making a greater surface area to become available for increased oxygen and nutrient consumption and release of cellular waste products in each cell of the body, thereby enhancing the overall health of the body at a cellular level.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

### Overview of the Device for Delivering Acoustic Vibrations

FIG. 1 illustrates a system or device 100 for delivering acoustic vibrations to a human body, in accordance with an embodiment of the present specification. The system 100 comprises a base assembly 102 and a tower assembly 104. The base assembly is configured to provide a horizontal platform 106 upon which a user may stand. In an embodiment, the length and width of the platform 106 ranges from 16″×20″ to 24″×36″. The platform 106 is elevated from the ground by a plurality of shock absorbent feet and a plurality of springs (shown in FIG. 2) which in embodiments, as discussed below, provide for an optimal platform 106 height.

When a user is standing on the base assembly 102, acoustic vibrations having frequencies ranging from 4 Hz to 20,000 Hz are delivered throughout the user's body, starting from the user's feet and extending upward. In various embodiments, the system 100 delivers multiple different frequencies comprising polychromatic, multi-tonal frequencies of sound throughout the user's body.

The tower assembly 104 is positioned proximate to the base assembly but preferably not fixedly attached to the base assembly 102, thereby allowing the base assembly 102 to move, i.e. vibrate, without shaking the tower assembly 104. The tower assembly comprises a housing 110 that extends vertically and has a height, depth and width of approximately 71″, 21″ and 34″ respectively. In embodiments, the housing 110 either comprises or is coupled to one or more of: a computing device (not shown in FIG. 1) comprising at least one processor, a memory (not shown in FIG. 1) storing programmatic instructions, and programmatic instructions that, when executed, generates at least one graphical user interface (not shown in FIG. 1) which may be displayed on screen 112 through which a user may control the system 100; one or more optional speakers 114, and one or more handle bars 116.

In various embodiments, acoustic waves are delivered into the user's body via at least one of or a combination of the base assembly 102, the speakers 114 of the tower assembly 104, and cell exciters that comprise contact pads electrically connected to the tower assembly 104, thereby providing multiple points of acoustic generation and contact. Operationally, the device 100 is configured to generate, and deliver to a standing user, acoustic waves having frequencies ranging from 4 Hz to 20,000 Hz. More specifically, the base assembly 102 is configured to generate acoustic waves having frequencies in a range of 4 Hz to 5,000 Hz and the tower assembly 104 is configured to generate acoustic waves having frequencies up to 20,000 Hz. In embodiments, the base assembly is configured to move, or displace, vertically (upward and downward) a distance of at least ⅜ inch during operation. In various embodiments, the graphical user interface, and computing device of system 100, are configured to enable a user to select a mode for their desired outcome, such as but not limited to, weight loss, anti-inflammation, pain treatment, joint pain and recovery, and have the desired frequencies combined and automatically implemented by the device 100. Operationally, the acoustic waves generated from the base assembly 102 and speakers 114 may be combined to cause multiple different frequency ranges to be concurrently delivered to a standing user. For example, a user may select a “weight loss mode” or “muscle pain” mode via options visually presented on the screen 112, wherein such selection would direct the computer device to cause the base assembly 102 and/or speakers 114 to concurrently generate and deliver to the standing user as many as 100 different frequencies, and preferably 1-75 different frequencies, or any numerical increment therein, and wherein the frequencies range from 4 Hz to 20,000 Hz. Each of the aforementioned components will be discussed in greater detail below.

### Base Assembly

FIG. 2A is a perspective break-away view of the base assembly shown in FIG. 1, in accordance with an embodiment of the present specification. FIG. 2B is bottom perspective break-way view of the base assembly shown in FIG. 2A, in accordance with an embodiment of the present specification. Referring to FIGS. 2A and 2B, base assembly 200 comprises a top plate 202 and a bottom plate 204 which are coupled together by four bearings 206 and four shafts 208. Bearings 206 are attached to each of four corners 202a, 202b, 202c, and 202d (not shown) of the top plate 202 such that the shafts 208 provided at each of the corresponding four corners 204e, 204f, 204g, and 204h (not shown) of the bottom plate 204 fasten with or are connected via the bearings 206 and effectively couple top plate 202 with bottom plate 204, with a space 210 between said plates. In an embodiment, the height of space 210 ranges from 5 13/16″ (5.8125) to 6 6/16″ (6.375), and is preferably approximately 6.1875 inches.

In various embodiments, a linear motor design comprising a plurality of voice coils of varying sizes and a plurality of magnets is employed to deliver a wide range of frequencies through the bottom plate 204. The different sized magnets and coils are placed in different positions on the bottom steel plate 204 to optimize the generation of acoustic frequencies through a wider range than is possible by using a single size magnet coil design or even by using two coils of the same size.

Referring to FIGS. 2A and 2B, two voice coils 212, one voice coil 214 (which in an embodiment is larger than voice coils 212) and four springs 216a, 216b, 217a, 217b are positioned on the bottom plate 204, such that they are housed within space 210. In embodiments, each of voice coils 212 has a diameter ranging from approximately 3 inches to 3.5 inches and is preferably 3.1875 inches and voice coil 214 has a diameter ranging from 6 inches to 7.4375 inches, and is preferably 6.75 inches. In embodiments, two magnet assemblies 218 and one magnet assembly 220 are positioned on the top plate 202. In embodiments, each of the two magnet assemblies 218 has a diameter ranging from approximately 3 inches to 5 inches and is preferably approximately 4.5 inches. In embodiments, magnet assembly 220 has a diameter ranging from 5.5 inches to 9 inches and is preferably approximately 8.5 inches. As shown in FIG. 2B, the two magnet assemblies 218 and the one magnet assembly 220 are positioned on the top plate 202, such that when the top and bottom plates 202, 204 are coupled, the magnet assemblies 218 and the magnet assembly 220 are positioned over the voice coils 212, and the voice coil 214 respectively, in the space 210. In embodiments, the space 210 optimizes the coils 212, 214 in conjunction with the magnetic fields produced by the magnet assemblies 218, 220 for producing a maximum thrust through load ranges of 100 to 550 pounds. A plastic enclosure 222 encloses the plates 202, 204 and the space 210, such that in an assembled state a user may stand on top of the plastic enclosure 222 for receiving acoustic vibrations generated by the base assembly 200 and propagated through the user's body through the user's feet and up through the remainder of the user's body.

In embodiments, one or more thermistors (not shown in the FIGS.) are incorporated in a lower portion of the voice coils 212, 214 to detect the temperature of the coils and turn off the system 200 when said temperature rises above a predefined threshold level, which in an embodiment is 350 degrees Fahrenheit.

In some embodiments, the plates 202, 204 are made of materials that resonate acoustic vibrations, such as but not limited to steel, stainless steel, aluminum, bronze, brass, titanium, and tin. In an embodiment, top and bottom plates 202, 204 are fabricated from steel having a thickness of approximately 0.1875 inches. In other embodiments, the thickness of plates 202, 204 is such that it allows acoustic vibrations to propagate through and ranges from 0.125 inch to 0.5 inch or any numerical increment therein.

In various embodiments, each of the plates 202, 204 comprises a rim, running along the periphery or edge of the plates. In embodiments, the rim is a contiguous strip of a metallic material provided around the outer edges or periphery of plates 202, 204 for controlling resonation produced due to the voice coils and magnets. In an embodiment, a rim is fabricated from a strip of a material such as, but not limited to, steel having a width ranging from 3/16 inches to ⅛ inches, or any numerical increment therein. In embodiments, the strip is welded to the periphery or outer edge of plates 202, 204. In some embodiments, the strip may be welded at specific points around the edges of a plate to obtain the rim. In less preferred embodiments, the strip is bolted to the periphery or outer edge of plates 202, 204. In an embodiment, the rim may be created in each of the plates by pressing the edges of the plates into the required shape.

In various embodiments, it is desirable to have an optimal range of motion of the magnet assemblies 218, 220 around the coils 212, 214 to produce a desired range of acoustic frequencies. FIG. 2C is a block diagram illustrating the magnetic force fields coupling between a voice coil and a magnet in the base assembly, in accordance with an embodiment of the present specification. When top and bottom plates 250 of the base assembly are coupled, magnet 240 and voice coils 242 are positioned such that the voice coil 242 is completely aligned with the magnet 240 in order to obtain the required movement of the magnetic assemblies for generating the required acoustic frequencies. As shown in FIG. 2C the voice coil 242 is required to lie within the magnetic flux lines 244 of the magnet 240. Springs (shown in FIGS. 2A and 2B) are used to maintain the required thrust because if a user's weight is low, the voice coil 242 may not lie fully inside the perpendicular lines of the flux field 244, whereas if the user is of a heavier weight, the voice coil 242 may be compressed beyond the flux field 244. In various embodiments, a narrow range of linear displacement of the magnetic assemblies 218, 220 is required, which is achieved by using the springs 216a, 216b, 217a, 217b.

In embodiments, such as shown in FIG. 2A, four springs 216a, 216b, 217a, 217b (or two springs as shown in FIG. 3A, which will be described in detail below) are employed for obtaining a required thrust on the top plate 202, while the downward motion of the plate is limited to a predefined range.

In various embodiments, in order to obtain frequencies up to 20,000 Hz, a downward motion of the top plate 202 (and thereby the coils 212, 214) is preferably less than one-half of an inch, and in an embodiment, the preferred motion is approximately 0.375 of an inch. In embodiments, a downward motion of the top plate 202 of more than 0.375 of an inch may cause higher frequency acoustic vibrations which may become unpleasant for a user to withstand. In embodiments, springs having different compression factors may be employed to support different weight ranges. In some embodiments, a first tall and narrow spring may be employed to support a first predefined range of user weights and a second shorter and broader spring may be employed for supporting a second predefined range of user weights which is higher than the first weights. In an embodiment, the first spring and the second spring are collinear wherein the first spring is placed within the second spring. In embodiments where collinear springs are used, a taller spring is placed inside a shorter spring, such that the taller spring engages when a weight is paced on the base plate, and if the weight is large enough to compress the taller spring to a maximum thrust, the shorter spring engages and is compressed.

Referring to FIGS. 2A and 2B, springs 216a, 216b, 217a, 217b are used to maintain the ideal thrust while limiting the motion of the top plate 202 to 0.375 of an inch, even when users having different weights stand on the base assembly 200. In an embodiment, a variable rate spring is used that increases the resistive force as the weight on the top plate 202 increases. In embodiments, the height of the springs 216a, 216b is greater than the height of the springs 217a, 217b, while the diameters of the springs 216a, 216b is smaller than the diameters of the springs 217a, 217b. In an embodiment, the heights of the springs 216a, 216b is approximately 1.875 inches and the diameters of the springs 216a, 216b is approximately 2.5 inches. In an embodiment, the heights of the springs 217a, 217b is approximately 2.5 inches and the diameters of the springs 217a, 217b is approximately 1.75 inches. In various embodiments, the height difference between the springs 216a, 216b, and 217a, 217b is 0.5 of an inch when said springs are in a mounted state. In an embodiment, the taller and narrower springs 216a, 216b support users having weights up to approximately 300 pounds, while the larger diameter springs 217a, 217b are pressed to maintain a predefined thrust when the user weight exceeds 300 pounds.

In another embodiment, the first set of springs 216a, 216b have a height ranging from 1.625 inches to 2.5 inches and a diameter of 1.75 inches; and the second set of springs 217a, 217b have a height ranging from 1.5 inches to 1.875 inches and a diameter of 2.5 inches. In embodiments, the taller and narrower springs 216a, 216b are pressed when a user having a weight up to 300 pounds stands on the top plate 202, while the shorter springs 217a, 217b, which also have a larger diameter are engaged only when a user having a weight greater than approximately 180 pounds (and in an embodiment up to 550 pounds) stands on the top plate 202. Hence, in various embodiments, by using two (or more) number of springs of differing dimensions, the base assembly 200 is enabled to operate with user weights ranging from 100 to 550 pounds

In an embodiment, the springs are coated with a flexible coating material for preventing ringing of the springs. In an embodiment a coating material such as, but not limited to, nylon is used to minimize resonance and obtain a required thrust.

In various embodiments, the gauge of wire that is used to fabricate the springs determines the amount of resistance needed to optimize the acoustic frequencies obtained from the system. Thus, in an embodiment, the spring is made from spring steel wire

In embodiments, the base assembly 200 (and base assembly 300 shown in FIG. 3A, 3B) produces acoustic waves in the form of sine waves, which do not introduce harmonics in the desired acoustic frequencies and can be reversed in direction via a smooth transition, which will reduce the risk of brain stem injury and muscle tears.

In an embodiment as shown in FIG. 2A, 2B the larger magnet assembly 220 is placed at the center of the top plate 202 which couples with the larger voice coil 214 for enabling the plate to resonate at a low end (ranging from approximately 4 Hz to 1000 Hz) of the required frequency range. As shown in FIG. 2A, 2B the smaller two magnet assemblies 218 are placed towards the edge of the top plate 202 to resonate at frequencies ranging from approximately 4 Hz to 5000 Hz of the required frequency range.

Referring to FIGS. 1, 2A and 2B, in an embodiment, the system 100 comprises a detection system for determining if a user standing on the enclosure 222 during operation of the system 100 has stepped off the base assembly 200. If it is detected that the user is not standing on the enclosure 222 for a predefined period of time, the system 100 is shut off automatically. In an embodiment, the detection system is provided in the form of a motion sensor 105 on the tower assembly 104. In an embodiment the motion sensor 105 is configured to detect motion within 2 feet of the tower assembly 104.

FIG. 3A illustrates the base assembly shown in FIG. 1, in accordance with another embodiment of the present specification. FIG. 3B illustrates another view of the base assembly shown in FIG. 3A, in accordance with an embodiment of the present specification. Referring to FIGS. 3A and 3B, base assembly 300 comprises a bottom steel plate 302 having a rim 304 running along the edges or periphery of the plate 302 and a top steel plate 312 having a rim 314 running along the edges or periphery of the plate 312. The bottom steel plate 302 comprises four shafts 306, two voice coils 308, and two springs 310a, 310b. The top steel plate 312 comprises four linear bearings 316 for coupling with the shafts 306, and two magnet assemblies 318 which are coupled with the two voice coils 308 when the bottom steel plate 302 is coupled with the top steel plate 312. In embodiments, each of voice coils 308 has a diameter ranging from approximately 3 inches to 3.5 inches and is preferably 3.1875 inches. In embodiments, two magnet assemblies 318 are positioned on the top plate 312. In embodiments, each of the two magnet assemblies 318 has a diameter ranging from approximately 3 inches to 5 inches and is preferably approximately 4.5 inches. A plastic enclosure 320 encloses the steel plates 302, 312, such that, in an assembled state, a user may stand on top of the plastic enclosure 320 for receiving the acoustic vibrations generated by the base assembly 300.

In various embodiments, in order to obtain frequencies up to 20,000 Hz, a downward motion of the top plate 312 (and thereby the coils 308) is ideally approximately 0.375 of an inch. In embodiments, a downward motion of the top plate 312 of more than 0.375 of an inch may cause low frequency acoustic vibrations which may become unpleasant for a user to withstand. Springs 310a, 310b are used to maintain the required thrust while limiting the motion of the top plate 312 to 0.375 of an inch, even when users having different weights stand on the base assembly 300.

In embodiments, the base assembly 300 shown in FIGS. 3A and 3B comprises only two springs 310a, 310b as the base assembly 300 is designed for supporting users having lower weights than the users of the base assembly 200 having four springs 216a, 216b, 217a, 217b as shown in FIGS. 2A and 2B. In an embodiment, a user weight of up to 300 is supported by base assembly 300 while the base assembly 200 can support a user weight of up to 550 pounds.

### Tower Assembly

FIG. 4A illustrates a perspective view of the tower assembly shown in FIG. 1, in accordance with an embodiment of the present specification. FIG. 4B illustrates a back view of the tower assembly shown in FIG. 4A, in accordance with an embodiment of the present specification. The tower assembly 400 comprises an inner assembly 402 and an enclosing case, enclosure, or housing 404. During operation of the system of the present specification, the tower assembly 400 is tethered to the base assembly shown in FIGS. 2A, 2B, 3A, 3B by means of dampening feet, which are designed to fit into alignment holes 406 located within the tower assembly 400. In an embodiment, the dampening feet are made of rubber. The dampening feet enable the base assembly to be decoupled with the tower assembly, while still being tethered to the tower assembly 400, via alignment holes 406, isolating the vibration of the base assembly from the inner assembly 402 that contains electronic modules. This enhances the life of the electronics of tower assembly 400 and prevents connectors from vibrating loose which may cause equipment failure. The provision of the dampening feet on the bottom plate 204 and bottom plate 302 also enables rapid installation of the system wherein the base assembly is coupled with the dampening feet which fit into aligning holes 406 positioned in the tower assembly 400.

Inner assembly 402 is designed as an elongated tower and comprises a set of speakers 408 provided on either side of the tower as shown in FIG. 4A, 4B for delivering acoustic frequencies in the range of 20 Hz to 20,000 Hz to users. In embodiments, speakers 408 are also used to deliver music to users while at the same time delivering therapeutic acoustic frequencies. In embodiments, the speakers are positioned such that the acoustic delivered is targeted at the core of a user standing on a base assembly tethered to the tower assembly.

Inner assembly 402 further comprises a plurality of cell exciter pads (not shown in FIGS. 4A, 4B) which are pads delivering acoustic frequencies in a desired range. Cell exciters pads are coupled to the inner assembly 402 by means of cell exciter jack output 410. Cell exciter pads can be directly applied to the skin of a user for delivering the required acoustic frequencies to the user's body. In an embodiment, a cell exciter pad is made of a thermoplastic polymer such as but not limited to acrylonitrile butadiene styrene (ABS) and has a diameter of 2 inches and may be applied directly on the skin of the user's body for delivering acoustic frequencies to a localized region of the user's body.

Inner assembly 402 also comprises an amplifier 412 for amplifying the acoustic frequencies generated by a computing device 414. Referring to FIGS. 2A, 2B along with FIGS. 4A, 4B, the amplifier 412 delivers the power to the voice coils 212, 214 of the base assembly. In embodiments, 2 channels are provided in the amplifier 412 to allow for different intensities to be delivered to each of the 3″ voice coil 212 and the 6″ voice coil 214.

The computing device 414 comprising an audio chip integrated with the inner assembly 402 allowing users to select a sound frequency mode which would cause the audio chip coupled with an internal preamplifier, the amplifier 412, speakers 408 and the cell exciter jack output 410 to generate a predefined range of frequencies to be delivered to the user's body. In various embodiments, the computing device 414 causes generation of acoustic signals comprising multiple acoustic frequencies (ranging from 4 Hz to 20 kHz) simultaneously.

The enclosing case 404, which, in an embodiment, is made of plastic and encloses and protects the inner assembly 402, comprises a user interface (UI) 416 coupled with the computing device 414. In an embodiment, the UI 416 is a screen allowing users to select a sound frequency mode by displaying a list of predefined conditions. In embodiments, the UI 416 may also display other predefined information such as, but not limited to benefits of the predefined conditions.

The tower assembly 400 may be powered via connection to a power outlet using a power inlet provided in the enclosing case 404.

In an embodiment, the inner assembly 402 comprises an audio out capability that allows a user to take all the acoustic frequencies generated by the computing device 414 and connect them to an additional external device for delivering said sound frequencies.

### Sound Pod

FIG. 5 illustrates a sound pod in accordance with an embodiment of the present specification. Sound pod 500 comprises an enclosure 502 housing a base assembly 504 and a tower assembly 506 as described above for delivering therapeutic sound frequencies into the body of a user standing on the base assembly. In various embodiments, the enclosure 502 reflects the sound produced by the base assembly 504 and the tower assembly 506 back at the user making the sound more effective and increasing its therapeutic benefits. Enclosure 502 may be made sound proof, so as to not disturb the surroundings, especially when the sound pod 500 is placed in a gym, clinic, spa or other public areas. In an embodiment, enclosure 502 comprises an aluminum frame and one or more double-walled polycarbonate panels. In other embodiments, other transparent, non-resonating materials such as, but not limited to safety glass, tempered glass, glass pane, and Plexiglas may be used to make the enclosure 502. In various embodiments, the walls of the enclosure 502 are kept transparent so as to eliminate feelings of claustrophobia among users. In various embodiments, inner surfaces of the enclosure 502 are reflective for reflecting the sound delivered by the base and tower assemblies 504, 506.

In various embodiments, sound pod 500 comprises an inlet for importing oxygen into the enclosure 502, via an external oxygen source. The imported oxygen may be breathed in by users via nasal cannulas provided within the enclosure 502 for further therapeutic benefits. Users may inhale the oxygen while they are receiving acoustic frequencies through the base and tower assemblies. In various embodiments, sound pod 500 is ventilated. In an embodiment, the enclosure 502 comprises air vents placed into the base of the panels of the enclosure 502 and a fan incorporated in the roof of the enclosure 502. The fan helps draw cool air in through the base, up through the pod 500, and out the roof of the enclosure 502 of the sound pod 500. In an embodiment, the roof of the enclosure 502 comprises a honeycomb structured material which is sound absorbent and has a sound reflective surface. In an embodiment, the honeycomb structure is enclosed within two fiberglass sheets. The enclosure 502 comprises a door for entering said enclosure. In an embodiment, the door comprises strip magnets for creating a sound seal. In an embodiment a latch in combination with weather-proof sealants are used to ensure a required closure of the door. In various embodiments various non-resonant materials may be used to couple the panels and the gate of the enclosure 502.

FIG. 6 is a flowchart illustrating a method for delivering multi-frequency acoustic vibrations into the body of a user for increasing the voltage of the cells of the user's body, in accordance with an embodiment of the present specification. At step 602 the user stands on the base assembly. In an embodiment, where the system comprises a sound pod, the user enters the sound pod and steps onto the base assembly. At step 604 the user selects a treatment mode from among a plurality of treatment modes by using the user interface provided with the tower assembly tethered to the base assembly. In an embodiment, the user may select treatment modes such as, but not limited to a weight loss mode, a pain management mode, an anti-aging mode, a health and fitness mode, and an enhancing sports performance mode. In an embodiment, the user firstly selects an ‘auto’ mode for deliverance of predefined acoustic frequencies at an intensity and a time that is based on the overall conditioning of the user. At step 606, the user receives acoustic vibrations of predefined frequencies corresponding to the selection from the base assembly, wherein the acoustic vibrations enter the user's body via the feet of the user. At step 608, the user receives acoustic vibrations of predefined frequencies corresponding to the selection from at least one speaker coupled with the tower assembly, wherein the acoustic vibrations travel through air and enter the user's body. At step 610, the one or more cell exciter pads are attached with a portion of the user's body. At step 612, the user receives acoustic vibrations of predefined frequencies corresponding to the selection from the one or more cell exciter pads, wherein the acoustic vibrations are targeted to the specific portion of the user's body to which the cell exciter pads are attached.

FIG. 7A is a screenshot of a selection screen presented to a user for selecting a frequency mode, in accordance with an embodiment of the present specification. Screenshot 700 includes section 702 comprising a plurality of tabs in a menu for selection of a frequency mode of operation of the system, such as ‘auto’, ‘manual’, ‘essentials’, sports/recovery’ and ‘wellness’. Section 702 also comprises tabs such as ‘help, ‘about’ and ‘exit’, which when clicked enable a user to obtain help regarding system operation, information regarding the system and its uses, and exit the system, respectively. Specifically, screenshot 700 illustrates a screen that is presented to the user upon selecting the tab ‘auto’ 704 in the section 702, and comprises a section 706 for selection of an automated operation intensity/frequency of the system with frequencies ranging from 4 Hz to 35 Hz, comprising tabs such as ‘ultra’, ‘high’, ‘medium’, and ‘low’, which, when selected, enable the system to generate a predefined set of frequencies in the range of 4 Hz to 35 Hz which have higher or lower intensities corresponding to a selected tab, and displaying a corresponding intensity value on a display 716, provided in section 706. A display 708 displays the acoustic frequency automatically generated by clicking on one or more tabs in the sections 702 and 706. A start button 710 when clicked begins the operation of the system which can be paused or stopped by clicking on ‘pause’ button 712 and ‘stop’ button 714, respectively. Section 718 displays a diagrammatic view of a user's body showing the areas in which the user is wishes to receive or is receiving acoustic frequencies from the system. In embodiments, the user can select a body area for receiving acoustic frequencies. The frequencies delivered automatically vary by the area of the body selected and are in the range of 4 Hz to 35 Hz. Section 720 enables the user to select a time period (in minutes) for which the acoustic frequencies are delivered, wherein the display 722 displays the selected time period and displays a countdown of the time remaining. Upon display 722 reaching no time remaining, the auto selection stops and the other frequency panels enabled from FIGS. 7C, 7D and 7E are also stopped. Tabs 724 (run cell tunes) and 726 (run tru resonance) are provided for enabling the user to select other frequency panels and sound frequency modalities that are not included in FIGS. 7C, 7C and 7E. These other modalities include Ancient Healing Tones, Chakra, TBSW (Trilateral Balanced Sine Wave) Generator, and a client database. Programs 724, 726 automatically turn off after 2 minutes of non-use.

FIG. 7B illustrates a screen that is presented to the user upon selecting the tab ‘manual’ 730 in section 702, shown in FIG. 7A. Referring back to FIG. 7B, screenshot 750 comprises a section 702 comprising a plurality of tabs for selection of a frequency mode of operation of the system, such as ‘auto’, ‘manual’, ‘essentials’, sports/recover’ and ‘wellness’. Section 702 also comprises tabs such as ‘help, ‘about’ and ‘exit’, which when clicked enable a user to obtain help regarding system operation, information regarding the system and its uses, obtaining updates and exit the system, respectively. Screenshot 750 illustrates a screen that is presented to the user upon selecting the tab ‘manual’ 730 in the section 702, and comprises a slider-bar 732 for selection of an operation intensity of the system which when set by the user enables the system to generate a user-selected intensity from slider bar 732, and display a corresponding intensity value on a display 734. In an embodiment, the manual intensity control slider 732 provides a means for adjusting the intensity of the single selected frequency. Thus, by adjusting the intensity, a user is able to control the linear movement of the plate in a vertical plane within inaudible frequencies ranging from 4 Hz to 50 Hz, which exercise every cell in the body. A slider-bar 736 enables the user to manually set an acoustic frequency value from 4 Hz to 47 Hz which is then displayed on display 738. A slider-bar 740 enables the user to select a time period in minutes ranging from 5 minutes to 30 minutes and in 5 minute increments thereof for which the acoustic frequencies are delivered, wherein the display 742 displays the selected time period and counts down the remaining time. A start button 744 when clicked begins the operation of the system which can be paused or stopped by clicking on ‘pause’ button 746 and ‘stop’ button 748, respectively. Tabs 724 and 726 are provided for enabling the user to select other frequency panels and sound frequency modalities that are not included in FIGS. 7C, 7C and 7E. These other modalities include Ancient Healing Tones, Chakra, TBSW (Trilateral Balanced Sine Wave) Generator, and a client database. These programs 724 726 automatically turn off after 2 minutes of non-use.

FIG. 7C illustrates a screen that is presented to the user upon selecting the ‘essentials’ tab 762 in section 702, shown in FIG. 7A. Screenshot 760 comprises a section 702 comprising a plurality of tabs for selection of a frequency mode of operation of the system, such as ‘auto’, ‘manual’, ‘essentials’, sports/recover’ and ‘wellness’. Section 702 also comprises tabs such as ‘help, ‘about’ and ‘exit’, which when clicked enable a user to obtain help regarding system operation, information regarding the system and its uses, and exit the system, respectively. Screenshot 760 illustrates a screen that is presented to the user upon selecting the tab ‘essentials’ 762 in the section 702, and comprises a slider-bar 764 for selection of an operational intensity of the audio signals delivered to the base assembly 102 which when set by the user enables the system to vary the intensity of the audio of the selected predefined set of frequencies to suit the user and subsequently display a corresponding intensity value on a display 766. In an embodiment, slider bar 764 controls the volume to the plate of the frequency formulas, which are higher frequencies and generally audible to a human ear, while other controls in FIG. 7A control inaudible frequencies that exercise a majority of cells in the body and move the plate in a purely vertical plane.

A slider-bar 768 enables the user to change the intensity of the audio signals of the selected predefined set of frequencies delivered to the audio speakers 408 and cell exciters 410 which is then displayed by display 769. A slider-bar 761 enables the user to select a time period in minutes ranging from 5 minutes to 30 minutes and in 5 minute increments thereof which the acoustic frequencies would be delivered, and the display 763 displays the selected time period and counts down the time remaining. Screen 760 also comprises an ‘energize’ section 765, a ‘detox/recovery’ section 767, a ‘fitness’ section 781 and a ‘relax’ section 783. In an embodiment, the sub-modes are capable of operating in frequencies, or a set of frequency formulas representing a combination of different frequencies, ranging from 4 Hz to 20,000 Hz. The ‘energize’ section 765 comprises tabs such as ‘dynamic warm up’, ‘stretch and flex’, ‘mood elevation’, and ‘fatigue’, each of which when selected by a user enable the system to generate a predefined set of frequencies corresponding to the selected tab and deliver the generated frequencies to the user. The ‘detox/recovery’ section 767 comprises tabs such as ‘lymph detox’, ‘detox’, ‘constipation’, ‘muscle pain’, and ‘pain management’, each of which when selected by a user enable the system to generate a predefined set of frequencies corresponding to the selected tab and deliver the generated frequencies to the user. The ‘fitness’ section 781 comprises tabs such as ‘weight loss’, ‘core’, ‘children’, ‘men’, ‘women’ and ‘elderly’, each of which when selected by a user enable the system to generate a predefined set of frequencies corresponding to the selected tab and deliver the generated frequencies to the user. The ‘relax’ section 783 comprises tabs such as ‘massage’, ‘stretch’, ‘stress relief’, ‘cool down’ and ‘insomnia’, each of which when selected by a user enable the system to generate a predefined set of frequencies corresponding to the selected tab and deliver the generated frequencies to the user. The frequency is in a range of 4 Hz to 20,000 Hz and is unique for each mode. A stop button 785 when pressed, stops the deliverance of said frequencies to the user.

FIG. 7D illustrates a screen 790 that is presented to the user upon selecting the tab ‘sports/recovery’ 792 in the section 702, shown in FIG. 7A. Screenshot 790 comprises a section 702 comprising a plurality of tabs for selection of a frequency mode of operation of the system, such as ‘auto’, ‘manual’, ‘essentials’, sports/recover’ and ‘wellness’. Section 702 also comprises tabs such as ‘help, ‘about’ and ‘exit’, which when clicked enable a user to obtain help regarding system operation, information regarding the system and its uses, and exit the system, respectively. Screenshot 790 illustrates a screen that is presented to the user upon selecting the tab ‘sports/recovery’ 792 in the section 702, and comprises a slider-bar 794 for selection of an operation intensity of frequencies delivered to the base assembly 102 which when set by the user enable the system to generate a predefined set of frequencies, and displaying a corresponding intensity value on a display 796. A slider-bar 798 enables the user to change the intensity of the audio signals or the sound volume of the selected predefined set of frequencies delivered to the audio speakers 408 and cell exciters 410 which is then displayed by display 799. A slider-bar 791 enables the user to select a time period in minutes ranging from 5 minutes to 30 minutes and in 5 minutes increments thereof, for which the acoustic frequencies would be delivered, and the display 793 displays the selected time period. Screen 790 also comprises a ‘warm up’ section 795, a ‘conditioning’ section 797, a ‘rehab’ section 7010 and a ‘recovery’ section 7011. The ‘warm up’ section 795 comprises tabs such as ‘dynamic warm up’, ‘flexibility’, and ‘energy boost’, each of which when selected by a user enable the system to generate a predefined set of frequencies corresponding to the selected tab and deliver the generated frequencies to the user. The ‘conditioning’ section 797 comprises tabs such as ‘women’, ‘men’ and ‘pain management’, each of which when selected by a user enable the system to generate a predefined set of frequencies corresponding to the selected tab and deliver the generated frequencies to the user. The ‘rehab’ section 7010 comprises tabs such as ‘upper body’ ‘core’, ‘lower body’ and ‘muscle pain’, each of which when selected by a user enable the system to generate a predefined set of frequencies corresponding to the selected tab and deliver the generated frequencies to the user. The ‘recovery’ section 7011 comprises tabs such as ‘relax’, ‘dynamic cool down’ and ‘inflammation’, each of which when selected by a user enable the system to generate a predefined set of frequencies corresponding to the selected tab and deliver the generated frequencies to the user. A stop button 7012 when pressed, stops the deliverance of said frequencies to the user.

FIG. 7E illustrates a screen 7020 that is presented to the user upon selecting the tab ‘wellness’ 7022 in the section 702, shown in FIG. 7A. Screenshot 7020 comprises a section 702 comprising a plurality of tabs for selection of a frequency mode of operation of the system, such as ‘auto’, ‘manual’, ‘essentials’, sports/recover’ and ‘wellness’. Section 702 also comprises tabs such as ‘help, ‘about’ and ‘exit’, which when clicked enable a user to obtain help regarding system operation, information regarding the system and its uses, and exit the system, respectively. Screenshot 7020 illustrates a screen that is presented to the user upon selecting the tab ‘wellness’ 7022 in the section 702, and comprises a slider-bar 7026 for selection of an operation intensity of frequencies delivered to the base assembly 102 which when set by the user enable the system to generate a predefined set of frequencies, and displaying a corresponding intensity value on a display 7024. A slider-bar 7028 enables the user to change the intensity of the audio signals of the selected predefined set of frequencies delivered to the audio speakers 408 and cell exciters 410 which level is then displayed by display 7029. A slider-bar 7021 enables the user to select a time period in minutes ranging from 5 minutes to 30 minutes and in 5 minute increments thereof for which the acoustic frequencies would be delivered, and the display 7023 displays the selected time period. Screen 7020 also comprises a ‘general’ section 7025, a ‘detox’ section 7027, a ‘exercise’ section 7031 and a ‘rehab’ section 7032. The ‘general’ section 7025 comprises tabs such as ‘bone density’, ‘hormonal imbalance’, ‘mood elevation’ and ‘numbness’, each of which when selected by a user enable the system to generate a predefined set of frequencies corresponding to the selected tab and deliver the generated frequencies to the user. The ‘detox’ section 7027 comprises tabs such as ‘lymphatic’, ‘chemical detox’, ‘edema’, ‘constipation’ and ‘pain management’, each of which when selected by a user enable the system to generate a predefined set of frequencies corresponding to the selected tab and deliver the generated frequencies to the user. The ‘exercise’ section 7031 comprises tabs such as ‘dynamic warm up’ ‘general exercise’, ‘weight loss’, ‘stretch and flex’, and ‘dynamic cool down’, each of which when selected by a user enable the system to generate a predefined set of frequencies corresponding to the selected tab and deliver the generated frequencies to the user. The ‘rehab’ section 7032 comprises tabs such as ‘recovery’, ‘muscle pain’ ‘back problems’, ‘knee pain’ and ‘joint pain’, each of which when selected by a user enable the system to generate a predefined set of frequencies corresponding to the selected tab and deliver the generated frequencies to the user. A stop button 7033 when pressed, stops the deliverance of said frequencies to the user.

### Exemplary Use Case Scenarios

In various embodiments, the system of the present specification may be used for a plurality of purposes, which are described in greater detail below.

### Enhancing Sports Performance

The system of the present specification may be used for enhancing sports performance of an athlete by vibrating the cells of the athlete's body. Such vibrations lead to increasing cellular vitality and voltage of the cells which correspondingly leads to increase in parameters such as the athlete's vertical jump height, jump length, speed, explosive power, coordination, balance, stamina, growth hormone production, testosterone, and speed of healing from sports related injuries. The use of the present systems further leads to a decrease in the athlete's recovery time, reduction of lactic acid accumulation, reduction of stress fractures and occurrence of injuries.

Referring to FIGS. 7A and 7C, in an exemplary scenario a user clicks on ‘auto’ tab 704 shown in screenshot 700, along with selecting the whole body for receiving acoustic frequencies from the display 718 with a medium or high intensity selected from section 706 for a selected time period of 20 minutes from the slider bar 720. Upon receiving such selection, the system automatically delivers frequencies ranging from 4 Hz to 35 Hz stimulating all the cells in the user's body increasing their cell voltage, while also increasing blood circulation and stimulating lymph drainage in the user's body. Next, the user clicks on ‘essentials’ tab 762 shown in screenshot 760 along with selecting “muscle pain” tab from the ‘detox/recovery’ section 767. This selection causes the system to specifically stimulate muscles cells in the user's body increasing their cellular voltage, and releasing lactic acid, causing the muscles to recover faster. In an embodiment, a plurality of frequencies such as, but not limited to 40 Hz, 45.8 Hz, 80 Hz, 82.5 Hz, 111 Hz, 112.5 Hz, 115 Hz, 116.2 Hz, 160 Hz, 160.5 Hz, 240 Hz, 250 Hz, 320 Hz, 324 Hz, 2720 Hz and 6000 Hz are delivered to the user.

### Promoting Health and Fitness and Enabling Weight Loss

The system of the present specification may be used for promoting health and fitness of users and aiding in weight loss by vibrating the cells of the users. The present system helps shorten recovery, releases lactic acid build up in a user's muscles, helps reduce and release that stubborn body fat, reduces cellulite, helps tighten and tone muscle, and improves the user's training results.

Referring to FIGS. 7A and 7C, in an exemplary scenario a user desiring weight loss clicks on ‘auto’ tab 704 shown in screenshot 700, along with selecting the whole body for receiving acoustic frequencies from the display 718 with a low or medium intensity selected from section 706 for a selected time period of 20 minutes from the slider bar 720. Upon receiving such selection, the system automatically delivers frequencies ranging from 4 Hz to 35 Hz stimulating all the cells in the user's body increasing their cell voltage, while also increasing blood circulation and stimulating lymph drainage in the user's body. Next, the user clicks on ‘essentials’ tab 762 shown in screenshot 760 along with selecting “weight loss” tab from the ‘fitness’ section 781. This selection causes the system to deliver predefined acoustic frequencies to the user's body enabling weight loss. In an embodiment, cell exciter pads may be placed on the skin of the user in an area of specific interest, which may, for example, be the belly region.

The specific plurality of frequencies delivered to the user's body result in lipolysis and cavitation of fat cells. A study to validate this claim was performed with 28 participants who used the system of the present specification for 1 month to evaluate the changes. The participants were instructed not to change their eating or exercise habits during this period. Objective and subjective measures were assessed at the beginning and the end of the study. The average results obtained are as follows: weight loss was 4.1 lbs.; changes in inches −13.3″; systolic blood pressure −3.9 mmHg; diastolic blood pressure −3.4 mmHg; heart rate −1.6 bpm; pain −31.3%; stress −35.6%; sleep improvement 20.9%; depression −25.5%; fatigue −24.5%. The plurality of frequencies used were 73 Hz, 80 Hz, 92 Hz, 95 Hz, 96.1 Hz, 160 Hz, 295.8 Hz, 302 Hz, 412 Hz, 414 Hz, 465 Hz, 528 Hz, 530.98 Hz, 728 Hz, 742 Hz, 787 Hz, 800 Hz, 803.5 Hz, 886 Hz, 3176 Hz, 5000 Hz, 5007.83 Hz, 10000 Hz and 16000 Hz.

### Aiding Injury Recovery and Rehabilitation

The system of the present specification may be used for aiding recovery and rehabilitation of users after various kinds of injuries by vibrating the cells of an injured body and increasing the voltage of the cells. The present system enables speeding up of the recovery time of an injured body. This is due to many factors that directly affect the area of injury; increase of circulation, reduction of inflammation, increase of cell voltage and enhancement of cellular repair.

Referring to FIGS. 7A and 7D, in an exemplary scenario a user seeking recovery from an injury clicks on ‘auto’ tab 704 shown in screenshot 700, along with selecting the whole body for receiving acoustic frequencies from the display 718 with a low, medium or high intensity selected from section 706, for a selected time period of 20 minutes from the slider bar 720. Upon receiving such selection, the system automatically delivers frequencies ranging from 4 Hz to 35 Hz stimulating all the cells in the user's body increasing their cell voltage, while also increasing blood circulation and stimulating lymph drainage in the user's body. Next, the user clicks on ‘sports/recovery’ tab 792 shown in screenshot 790 along with selecting ‘muscle pain’ tab from the ‘rehab’ section 7010; and ‘inflammation’ tab from the ‘recovery’ section 7011. One or more cell exciter pads are applied to the region of injury on the user's body. This selection causes the system to deliver predefined acoustic frequencies to the user's body aiding and enabling recovery from the injury. In an embodiment, a plurality of frequencies such as, but not limited to 40 Hz, 45.8 Hz, 80 Hz, 82.5 Hz, 111 Hz, 112.5 Hz, 115 Hz, 116.2 Hz, 121.5 Hz, 160 Hz, 160.5 Hz, 240 Hz, 250 Hz, 320 Hz, 324 Hz, 2720 Hz, 3000 Hz, 5000 Hz and 6000 Hz are delivered to the user.

### Providing Anti Aging Benefits

The system of the present specification may be used for providing anti-aging benefits and enhancing the quality of life of older patients. The vibrations delivered by the present system aid in enhancing balance of older patients thereby reducing fall risks. Use of the present system increases the lower extremity muscle strength and power in the older population and enhances their quality of life.

Other benefits may include balance, bone density, hormonal corrections, motor neuron efficacy, oxygen uptake, and improvement in other physiological factors.

The above examples are merely illustrative of the many applications of the system of present specification. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

We claim:

1. A system configured to deliver multi-frequency acoustic vibrations to cells of a user's body, the system comprising:
a base assembly comprising a top plate, a bottom plate, a space defined by the top plate and the bottom plate, one or more voice coils positioned in the space, one or more magnets positioned in the space, and one or more springs positioned in the space, wherein the one or more springs is configured to maintain a predefined thrust range between the top plate and the bottom plate, wherein each of the one or more voice coils is aligned with one of the one or more magnets such that the one or more voice coils lies within a magnetic flux of the one or more magnets to thereby produce a first portion of the multi-frequency acoustic vibrations;
a vertically extending assembly tethered to the base assembly, wherein the vertically extending assembly comprises at least one speaker configured to produce a second portion of the multi-frequency acoustic vibrations; and
one or more cell exciter pads coupled with the vertically extending assembly and configured to attach to a portion of the user's body to deliver a third portion of the multi-frequency acoustic vibrations.

2. The system of claim 1, wherein the acoustic vibrations comprise acoustic waves of frequencies ranging from 4 to 20,000 Hz.

3. The system of claim 1, wherein the acoustic vibrations comprise polychromatic and multi-tonal acoustic frequencies.

4. The system of claim 1, further comprising a casing enclosing the top plate the bottom plate, wherein the casing is adapted to receive a user standing thereon.

5. The system of claim 1, wherein the top plate is coupled with the bottom plate through a plurality of shafts and bearings.

6. The system of claim 1, wherein the one or more magnets comprise a first magnet, a second magnet, and a third magnet coupled to the top plate, wherein the third magnet is larger than the first magnet and the second magnet, wherein the one or more voice coils comprise a first voice coil, a second voice coil, and a third voice coil coupled to the bottom plate, and wherein the third voice coil is larger than the first voice coil and the second voice coil.

7. The system of claim 1 wherein the one or more magnets comprise only two magnets, wherein the two magnets are coupled with the top plate, wherein the one or more voice coils comprise only two voice coils, and wherein the two voice coils are coupled with the bottom plate.

8. The system of claim 1, wherein the one or more springs comprise two or more springs each having a different diameter and a different length to thereby provide a variable thrust based on the user's weight.

9. The system of claim 1, wherein the top plate and the bottom plate comprise steel and have a thickness of 3/16 inches.

10. The system of claim 1, wherein the top plate and bottom plate are each made of at least one of stainless steel, aluminum, bronze, brass, titanium, or tin.

11. The system of claim 1, wherein each of the top plate and the bottom plate comprise a rim extending all along edges of the top plate and the bottom plate and has a thickness ranging from 3/16 to ⅛ inches.

12. The system of claim 1, wherein the vertically extending assembly further comprises a casing enclosing said vertically extending assembly, wherein an outer surface of the casing comprises a reflective material adapted to reflect the second portion of the multi-frequency acoustic vibrations produced by the vertically extending assembly back to the user.

13. The system of claim 1, wherein the vertically extending assembly is tethered to the base assembly by one or more dampening feet extending on a first side below the base assembly and on a second side being coupled with the vertically extending assembly.

14. The system of claim 1, further comprising a computing device comprising an audio chip coupled with an amplifier coupled with the at least one speaker and the one or more cell exciter pads adapted to enable the user to select a treatment mode from among a plurality of treatment modes, and wherein the computing device is configured to cause the audio chip to generate the multi-frequency acoustic vibrations based upon said selection.

15. The system of claim 14, wherein the vertically extending assembly further comprises a user interface enabling the user to make the selection.

16. The system of claim 14, wherein the plurality of treatment modes comprises at least one of a weight loss mode, a pain management mode, an anti-aging mode, a health and fitness mode, or an enhancing sports performance mode.

17. The system of claim 14, wherein the plurality of treatment modes comprises at least seven hundred predefined combinations of the multi-frequency acoustic vibrations.

18. The system of claim 1, further comprising a sound pod enclosing the base assembly and the vertically extending assembly, wherein the sound pod comprises a roof, enclosing walls and a door adapted to allow the user to enter the sound pod, wherein at least the walls and door comprise a transparent, non-resonating material.

19. The system of claim 18, wherein the roof comprises a non-resonating material.

20. The system of claim 18, wherein the walls and door comprise double-walled polycarbonate panels supported by aluminum frames.

21. The system of claim 18, further comprising an oxygen source, wherein the sound pod comprises an inlet adapted to receive oxygen from the oxygen source, and a nasal cannula adapted to direct the oxygen to the user.

22. The system of claim 18, wherein the sound pod further comprises a fan and a plurality of air vents, wherein the plurality of air vents are positioned in a base of at least one of the walls and wherein the fan is positioned on or in the roof.

23. A method of delivering multi-frequency acoustic vibrations into a body of a user, the method comprising:
providing an acoustic generation system, comprising:
a base assembly comprising a top plate, a bottom plate, a space defined by the top plate and the bottom plate, one or more voice coils positioned in the space, one or more magnets positioned in the space, and one or more springs positioned in the space, wherein the one or more springs is configured to maintain a predefined thrust range between the top plate and the bottom plate, wherein each of the one or more voice coils is aligned with one of the one or more magnets such that the one or more voice coils lies within a magnetic flux of the one or more magnets to thereby produce a first portion of the multi-frequency acoustic vibrations;
a vertically extending assembly tethered to the base assembly, wherein the vertically extending assembly comprises at least one speaker configured to produce a second portion of the multi-frequency acoustic vibrations; and
one or more cell exciter pads coupled with the vertically extending assembly and configured to attach to a portion of the user's body to deliver a third portion of the multi-frequency acoustic vibrations;
instructing the user to stand on the base assembly;
selecting a treatment mode from among a plurality of treatment modes using an interface in data communication with the acoustic generation system;
generating one or more of the multi-frequency acoustic vibrations based on the selected treatment mode; and
applying the one or more of the multi-frequency acoustic vibrations to the user's feet via the base assembly, the user's body through air from the at least one speaker, or the user's body via the one or more cell exciter pads attached to a portion of the user's body.

24. The method of claim 23 wherein the multi-frequency acoustic vibrations comprise acoustic of frequencies ranging from 4 to 20,000 Hz.
